(19) United States
(12) Patent Application Publication
(10) Pub. No.: US 2008/0138654 A1
(43) Pub. Date: Jun. 12, 2008

(54) Electroluminescent Materials and Devices

(75) Inventors: Poopathy Kathirgamanathan, North Harrow (GB); Alexander Kit Lay, Reading (GB); Muttulingam Kumaraverl, London (GB); Subramaniam Ganeshamurugan, London (GB)

Correspondence Address: David Silverstein;Andover-IP-Law, 44 Park Street, Suite 300, Andover, MA, 01810, US

(73) Assignee: OLED-T Limted, Enfield (GB)

(21) Appl. No.: 11/792,167

(22) PCT Filed: Dec. 6, 2005
(86) PCT No.: PCT/GB05/04673

§ 371 Date: Jun. 25, 2007

(30) Foreign Application Priority Data
Dec. 6, 2004 (GB) 0426674.8

(51) Int. Cl. C09K 11/06; C09K 11/00; C07D 223/18; C07F 5/02; C07C 211/55; C07C 211/61

(52) U.S. Cl. 428/690; 540/588; 546/13; 564/433; 564/427

(57) ABSTRACT

A hole transporting or hole conducting material for use in electroluminescent devices is a diamino dianthracene.

The present invention relates to hole transporting or hole conducting materials for use in electroluminescent devices.

Materials which emit light when an electric current is passed through them are well known and used in a wide range of display applications. Liquid crystal devices and devices which are based on inorganic semiconductor systems are widely used; however these suffer from the disadvantages of high energy consumption, high cost of manufacture, low quantum efficiency and the inability to make flat panel displays.

Organic polymers have been proposed as useful in electroluminescent devices, but it is not possible to obtain pure colours, they are expensive to make and have a relatively low efficiency.

Another compound which has been proposed is aluminium quinolate, but this requires dopants to be used to obtain a range of colours and has a relatively low efficiency.

Patent application WO98/58037 describes a range of lanthanide complexes which can be used in electroluminescent devices which have improved properties and give better results. Patent Applications PCT/GB98/01773, PCT/GB99/03619, PCT/GB99/04030, PCT/GB99/04028, PCT/GB00/00268 describe further electroluminescent complexes, structures and devices using rare earth chelates.

Typical electroluminescent devices which are commonly referred to as optical light emitting diodes (OLEDS) comprise an anode, normally of an electrically light transmitting material, a layer of a hole transporting material, a layer of the electroluminescent material, a layer of an electron injecting or transporting material and a metal cathode.

U.S. Pat. No. 5,128,587 discloses an electroluminescent device which consists of an organometallic complex of rare earth elements of the lanthanide series sandwiched between a transparent electrode of high work function and a second electrode of low work function with a hole conducting layer interposed between the electroluminescent layer and the transparent high work function electrode and an electron conducting layer interposed between the electroluminescent layer and the electron injecting low work function anode. The hole conducting layer and the electron conducting layer are stated as being required to improve the working and efficiency of the device. The hole conducting or transportation layer serves to transport holes and to block the electrons, thus preventing electrons from moving into the electrode without recombining with holes. The electron conducting or transporting layer serves to transport electrons and to block the holes, thus preventing holes from moving into the electrode without recombining with holes. The recombination of carriers therefore mainly or entirely takes place in the emitter layer.

As described in U.S. Pat. No. 6,333,521 this mechanism is based upon the radiative recombination of a trapped charge. Specifically, this patent describes OLEDs which are comprised of at least two thin organic layers between an anode and a cathode. The material of one of these layers is specifically chosen based on the material's ability to transport holes, a “hole transporting layer” (HTL), and the material of the other layer is specifically selected according to its ability to transport electrons, an “electron transporting layer” (ETL). With such a construction, the device can be viewed as a diode with a forward bias when the potential applied to the anode is higher than the potential applied to the cathode. Under these bias conditions, the anode injects holes (positive charge carriers) into the HTL, while the cathode injects electrons into the ETL. The portion of the luminescent medium adjacent to the anode thus forms a hole injecting and transporting zone while the portion of the luminescent medium adjacent to the cathode forms an electron injecting and transporting zone. The injected holes and electrons each migrate toward the oppositely charged electrode. When an electron and hole localise on the same molecule, a Frenkel exciton is formed. These excitons are trapped in the material which has the lowest energy. Recombination of the short-lived excitons may be visualized as an electron dropping from its conduction potential to a valence band, with relaxation occurring, under certain conditions, preferentially via a photoemissive mechanism.

The materials that function as the ETL or HTL of an OLED may also serve as the medium in which exciton formation and electroluminescent emission occur. Such OLEDs are referred to as having a “single heterostructure” (SH). Alternatively, the electroluminescent material may be present in a separate emissive layer between the HTL and the ETL in what is referred to as a “double heterostructure” (DH).

In a single heterostructure OLED, either holes are injected from the HTL into the ETL where they combine with electrons to form excitons, or electrons are injected from the ETL into the HTL where they combine with holes to form excitons. Because excitons are trapped in the material having the lowest energy gap, and commonly used ETL materials generally have smaller energy gaps than commonly used HTL materials, the emissive layer of a single heterostructure device is typically the ETL. In such an OLED, the materials used for the ETL and HTL should be chosen such that holes can be injected efficiently from the HTL into the ETL. Also, the best OLEDs are believed to have good energy level alignment between the highest occupied molecular orbital (HOMO) levels of the HTL and ETL materials.

In a double heterostructure OLED, holes are injected from the HTL and electrons are injected from the ETL into the separate emissive layer, where the holes and electrons combine to form excitons.

Various compounds have been used as HTL materials or ETL materials. HTL materials mostly consist of triaryl amines in various forms which show high hole mobilities (˜10⁻³ cm²/Vs). There is somewhat more variety in the ETLs used in OLEDs. Aluminum tris(8-hydroxyquinolate) (Alq₃) is the most common ETL material, and others include zirconium quinolate, hafnium quinolate, oxidiazol, triazol, and triazine.

A well documented cause of OLED failure is thermally induced deformation of the organic layers (e.g. melting, crystal formation, thermal expansion, etc.). This failure mode can be seen in the studies that have been carried out with hole transporting materials, K. Naito and A. Miura, J. Phys. Chem. (1993), 97, 6240-6248; S. Tokito, H. Tanaka, A. Okada and Y. Taga. Appl. Phys. Lett. (1996), 69, (7), 878-880; Y. Shirota, T Kobata and N. Noma, Chem. Lett. (1989), 1145-1148; T. Noda, I. Imae, N. Noma and Y. Shirota, Adv. Mater. (1997), 9, No. 3; E. Han, L. Do, M. Fujihira, H. Inada and Y. Shirota, J. Appl. Phys. (1996), 80, (6) 3297-701; T. Noda, H. Ogawa, N. Noma and Y. Shirota, Appl. Phys. Lett. (1997), 70, (6), 699-701; S. Van Slyke, C. Chen and C. Tang, Appl. Phys. Lett. (1996), 69, 15, 2160-2162; and U.S. Pat. No. 5,061,569.

In order to overcome this problem U.S. Pat. No. 6,333,521 discloses organic materials that are present as a glass, as opposed to a crystalline or polycrystalline form, which are disclosed for use in the organic layers of an OLED, since glasses are capable of providing higher transparency as well as producing superior overall charge carrier characteristics as compared with the polycrystalline materials that are typically produced when thin films of the crystalline form of the materials are prepared. However, thermally induced deformation of the organic layers may lead to catastrophic and irreversible failure of the OLED if a glassy organic layer is heated above its T_{g}. In addition, thermally induced deformation of a glassy organic layer may occur at temperatures lower than T_{g}, and the rate of such deformation may be dependent on the difference between the temperature at which the deformation occurs and T_{g}. Consequently, the lifetime of an OLED may be dependent on the T_{g} of the organic layers even if the device is not heated above T_{g}. As a result, there is a need for organic materials having a high T_{g} that can be used in the organic layers of an OLED.

However there is a general inverse correlation between the T_{g} and the hole transporting properties of a material, i.e., materials having a high T_{g} generally have poor hole transporting properties. Using an HTL with good hole transporting properties leads to an OLED having desirable properties such as higher quantum efficiency, lower resistance across the OLED, higher power quantum efficiency, and higher luminance.

We have now invented hole transporting compounds and devices incorporating them which reduce this problem.

According to the invention there is provided a hole transporting compound which comprises a diamino dianthracene of formula

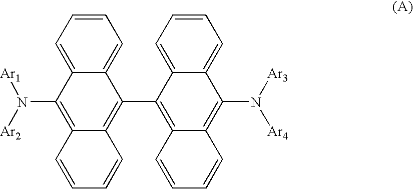

where Ar₁, Ar₂, Ar₃ and Ar₄ are the same or different substituted or unsubstituted aromatic groups including substituted or unsubstituted monocyclic, heterocyclic or polycyclic aromatic groups such as phenyl, naphthyl, phenanthrenyl etc.

The preferred groups Ar₁, Ar₂, Ar₃ and Ar₄ are substituted and unsubstituted phenyl, bisphenyl, naphthyl, anthracenyl, heterocyclic and fused rings where Ar₁ and Ar₂ or Ar₃ and Ar₄ form a heterocyclic ring with the nitrogen atom. The substituents can be selected from hydrogen, and alkyl, aliphatic, aromatic and heterocyclic alkoxy, aryloxy and carboxy groups, such as c1-4 alkyl e.g. t-butyl and heterocyclic groups such as carbazole and trimethyl fluorine.

Examples of groups Ar₁, Ar₂, Ar₃ and Ar₄ are

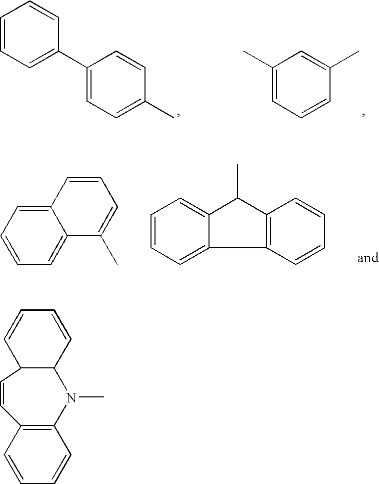

The invention also provides an electroluminescent device which comprises (i) a first electrode, (ii) a layer of a hole transporting layer which comprises a diamino dianthracene of formula (A) above, (iii) a layer of an electroluminescent material and (iv) a second electrode.

The thickness of the hole transporting layer is preferably 20 nm to 200 nm.

The electroluminescent material can be any electroluminescent compound such as a polymer electroluminescent compound, a small molecule electroluminescent compound such as a quinolate or a thioquinolate, e.g. aluminium quinolate, lithium quinolate, zirconium quinolate, hafnium quinolate, or an organometallic electroluminescent compound.

Electroluminescent compounds which can be used in the present invention are of general formula (Lα)ₙM where M is a rare earth, lanthanide or an actinide, Lα is an organic complex and n is the valence state of M.

Other organic electroluminescent compounds which can be used in the present invention are of formula

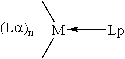

where Lα and Lp are organic ligands, M is a rare earth, transition metal, lanthanide or an actinide and n is the valence state of the metal M. The ligands Lα can be the same or different and there can be a plurality of ligands Lp which can be the same or different.

For example (L₁)(L₂)(L₃)(L . . . )M(Lp) where M is a rare earth, transition metal, lanthanide or an actinide and (L₁)(L₂)(L₃)(L . . . ) are the same or different organic complexes and (Lp) is a neutral ligand. The total charge of the ligands (L₁)(L₂)(L₃)(L . . . ) is equal to the valence state of the metal M. Where there are 3 groups Lα which corresponds to the III valence state of M the complex has the formula (L₁)(L₂)(L₃)M (Lp) and the different groups (L₁)(L₂)(L₃) may be the same or different.

Lp can be monodentate, bidentate or polydentate and there can be one or more ligands Lp.

Preferably M is metal ion having an unfilled inner shell and the preferred metals are selected from Sm(III), Eu(II), Eu(III), Tb(III), Dy(III), Yb(III), Lu(III), Gd (III), U(III), Tm(III), Ce (III), Pr(III), Nd(III), Pm(III), Ho(III), Er(III), Yb(III) and more preferably Eu(III), Tb(III), Dy(III), Gd (III), Er (III), Yt(III).

Further organic electroluminescent compounds which can be used in the present invention are of general formula (Lα)ₙM₁M₂ where M₁ is the same as M above, M₂ is a non rare earth metal, Lα is as above and n is the combined valence state of M₁ and M₂. The complex can also comprise one or more neutral ligands Lp so the complex has the general formula (Lα)ₙ M₁ M₂ (Lp), where Lp is as above. The metal M₂ can be any metal which is not a rare earth, transition metal, lanthanide or an actinide. Examples of metals which can be used include lithium, sodium, potassium, rubidium, caesium, beryllium, magnesium, calcium, strontium, barium, copper (I), copper (II), silver, gold, zinc, cadmium, boron, aluminium, gallium, indium, germanium, tin (II), tin (IV), antimony (II), antimony (IV), lead (II), lead (IV) and metals of the first, second and third groups of transition metals in different valence states e.g. manganese, iron, ruthenium, osmium, cobalt, nickel, palladium(II), palladium(IV), platinum(II), platinum(IV), cadmium, chromium. titanium, vanadium, zirconium, tantalum, molybdenum, rhodium, iridium, titanium, niobium, scandium, yttrium.

For example (L₁)(L₂)(L₃)(L . . . )M (Lp) where M is a rare earth, transition metal, lanthanide or an actinide and (L₁)(L₂)(L₃)(L . . . ) and (Lp) are the same or different organic complexes.

Further organometallic complexes which can be used in the present invention are binuclear, trinuclear and polynuclear organometallic complexes e.g. of formula (Lm)ₓ M₁←M₂(Ln)_{y} e.g.

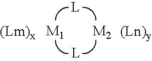

where L is a bridging ligand and where M₁ is a rare earth metal and M₂ is M₁ or a non rare earth metal, Lm and Ln are the same or different organic ligands Lα as defined above, x is the valence state of M₁ and y is the valence state of M₂.

In these complexes there can be a metal to metal bond or there can be one or more bridging ligands between M₁ and M₂ and the groups Lm and Ln can be the same or different.

By trinuclear is meant there are three rare earth metals joined by a metal to metal bond i.e. of formula

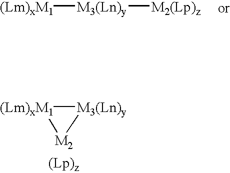

where M₁, M₂ and M₃ are the same or different rare earth metals and Lm, Ln and Lp are organic ligands Lα and x is the valence state of M₁, y is the valence state of M₂ and z is the valence state of M₃. Lp can be the same as Lm and Ln or different.

The rare earth metals and the non rare earth metals can be joined together by a metal to metal bond and/or via an intermediate bridging atom, ligand or molecular group.

For example the metals can be linked by bridging ligands e.g.

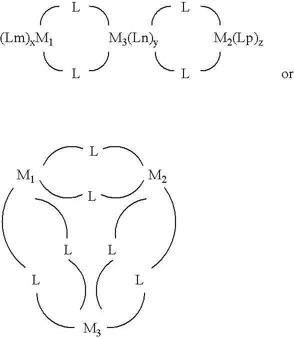

where L is a bridging ligand.

By polynuclear is meant there are more than three metals joined by metal to metal bonds and/or via intermediate ligands

M₁-M₂-M₃-M₄

or

M₁-M₂-M₄-M₃

or

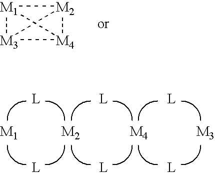

where M₁, M₂, M₃ and M₄ are rare earth metals and L is a bridging ligand.

Preferably Lα is selected from β diketones such as those of formulae

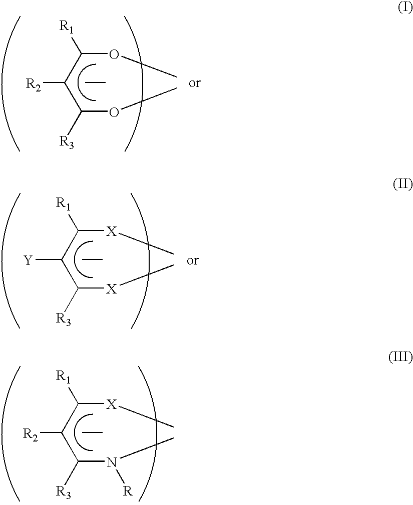

where R₁, R₂ and R₃ can be the same or different and are selected from hydrogen, and substituted and unsubstituted hydrocarbyl groups such as substituted and unsubstituted aliphatic groups, substituted and unsubstituted aromatic, heterocyclic and polycyclic ring structures, fluorocarbons such as trifluoryl methyl groups, halogens such as fluorine or thiophenyl groups; R₁, R₂ and R₃ can also form substituted and unsubstituted fused aromatic, heterocyclic and polycyclic ring structures and can be copolymerisable with a monomer e.g. styrene. X is Se, S or O, Y can be hydrogen, substituted or unsubstituted hydrocarbyl groups, such as substituted and unsubstituted aromatic, heterocyclic and polycyclic ring structures, fluorine, fluorocarbons such as trifluoryl methyl groups, halogens such as fluorine or thiophenyl groups or nitrile.

The beta diketones can be polymer substituted beta diketones and in the polymer, oligomer or dendrimer substituted β diketone the substituents group can be directly linked to the diketone or can be linked through one or more —CH₂ groups i.e.

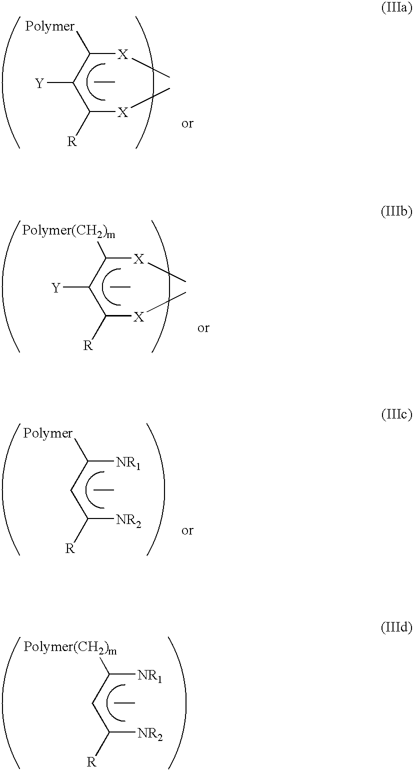

or through phenyl groups e.g.

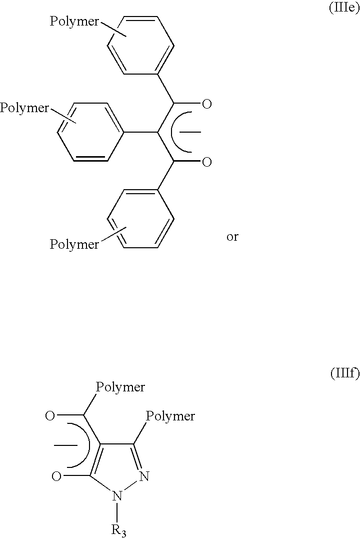

where “polymer” can be a polymer, an oligomer or a dendrimer, (there can be one or two substituted phenyl groups as well as three as shown in (IIIc)) and where R is selected from hydrogen, and substituted and unsubstituted hydrocarbyl groups such as substituted and unsubstituted aliphatic groups, substituted and unsubstituted aromatic, heterocyclic and polycyclic ring structures, fluorocarbons such as trifluoryl methyl groups, halogens such as fluorine or thiophenyl groups.

Examples of R₁ and/or R₂ and/or R₃ include aliphatic, aromatic and heterocyclic alkoxy, aryloxy and carboxy groups, substituted and substituted phenyl, fluorophenyl, biphenyl, phenanthrene, anthracene, naphthyl and fluorene groups alkyl groups such as t-butyl, heterocyclic groups such as carbazole.

Some of the different groups Lα may also be the same or different charged groups such as carboxylate groups so that the group L₁ can be as defined above and the groups L₂, L₃ . . . can be charged groups such as

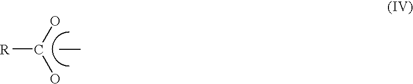

where R is R₁ as defined above or the groups L₁, L₂ can be as defined above and L₃ . . . etc. are other charged groups.

R₁, R₂ and R₃ can also be

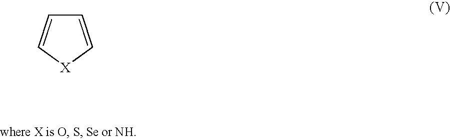

A preferred moiety R₁ is trifluoromethyl CF₃ and examples of such diketones are, benzoyltrifluoroacetone, p-chlorobenzoyltrifluoroacetone, p-bromotrifluoroacetone, p-phenyltrifluoroacetone, 1-naphthoyltrifluoroacetone, 2-naphthoyltrifluoroacetone, 2-phenathoyltrifluoroacetone, 3-phenanthoyltrifluoroacetone, 9-anthroyltrifluoroacetonetrifluoroacetone, cinnamoyltrifluoroacetone, and 2-thenoyltrifluoroacetone.

The different groups Lα may be the same or different ligands of formulae

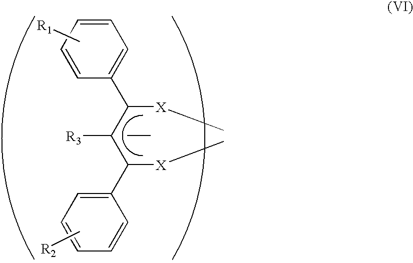

where X is O, S, or Se and R₁ R₂ and R₃ are as above.

The different groups Lα may be the same or different quinolate derivatives such as

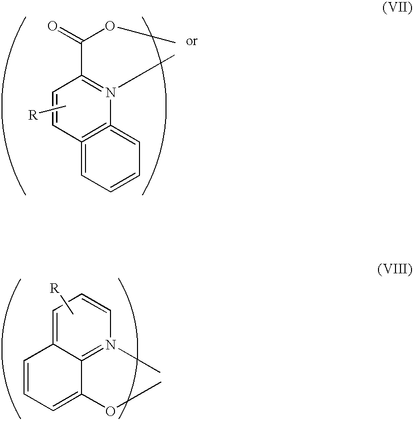

where R is hydrocarbyl, aliphatic, aromatic or heterocyclic carboxy, aryloxy, hydroxy or alkoxy e.g. the 8 hydroxy quinolate derivatives or

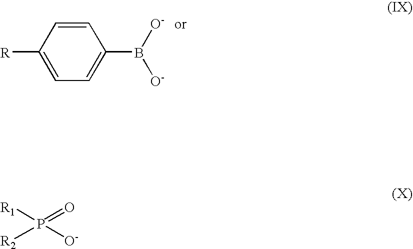

where R, R₁, and R₂ are as above or are H or F e.g. R₁ and R₂ are alkyl or alkoxy groups

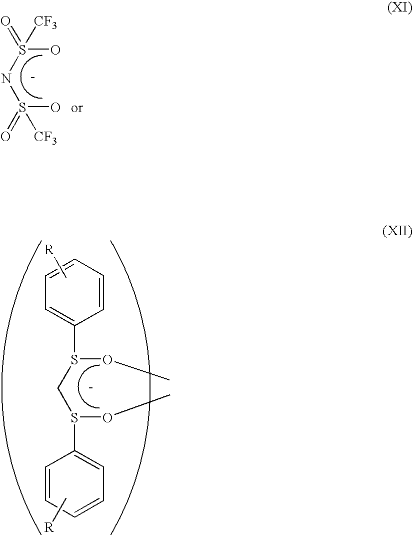

As stated above the different groups Lα may also be the same or different carboxylate groups e.g.

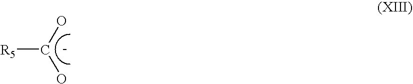

where R₅ is a substituted or unsubstituted aromatic, polycyclic or heterocyclic ring a polypyridyl group, R₅ can also be a 2-ethyl hexyl group so Ln is 2-ethylhexanoate or R₅ can be a chair structure so that Lₙ is 2-acetyl cyclohexanoate or Lα can be

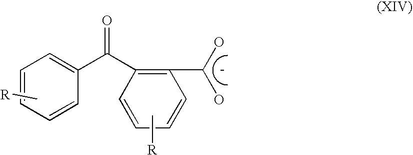

where R is as above e.g. alkyl, allenyl, amino or a fused ring such as a cyclic or polycyclic ring.

The different groups Lα may also be

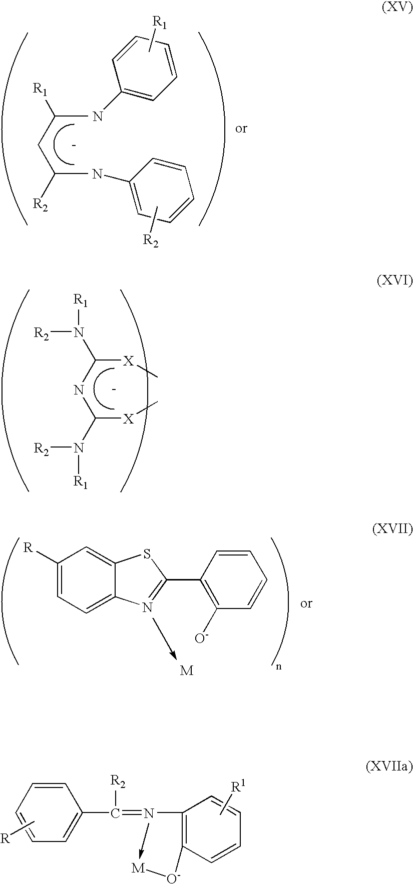

where R, R₁ and R₂ are as above.

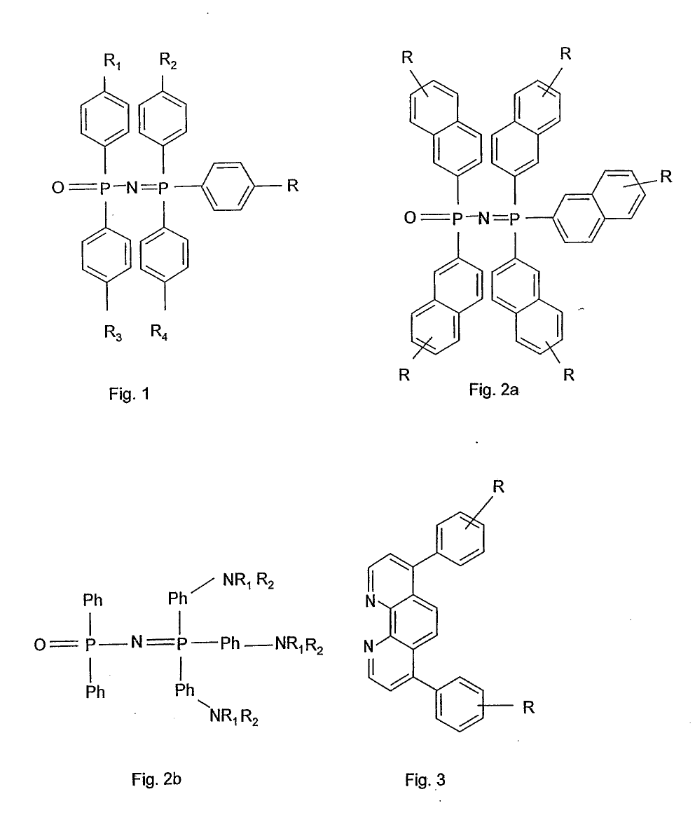

The groups Lₚ can be selected from

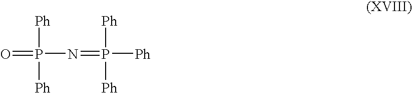

where each Ph which can be the same or different and can be a phenyl (OPNP) or a substituted phenyl group, other substituted or unsubstituted aromatic group, a substituted or unsubstituted heterocyclic or polycyclic group, a substituted or unsubstituted fused aromatic group such as a naphthyl, anthracene, phenanthrene or pyrene group. The substituents can be for example an alkyl, aralkyl, alkoxy, aromatic, heterocyclic, polycyclic group, halogen such as fluorine, cyano, amino, substituted amino etc. Examples are given in FIGS. 1 and 2 of the drawings where R, R₁, R₂, R₃ and R₄ can be the same or different and are selected from hydrogen, hydrocarbyl groups, substituted and unsubstituted aromatic, heterocyclic and polycyclic ring structures, fluorocarbons such as trifluoryl methyl groups, halogens such as fluorine or thiophenyl groups; R, R₁, R₂, R₃ and R₄ can also form substituted and unsubstituted fused aromatic, heterocyclic and polycyclic ring structures and can be copolymerisable with a monomer e.g. styrene. R, R₁, R₂, R₃ and R₄ can also be unsaturated alkylene groups such as vinyl groups or groups

—C—CH₂═CH₂—R

where R is as above.

Lₚ can also be compounds of formulae

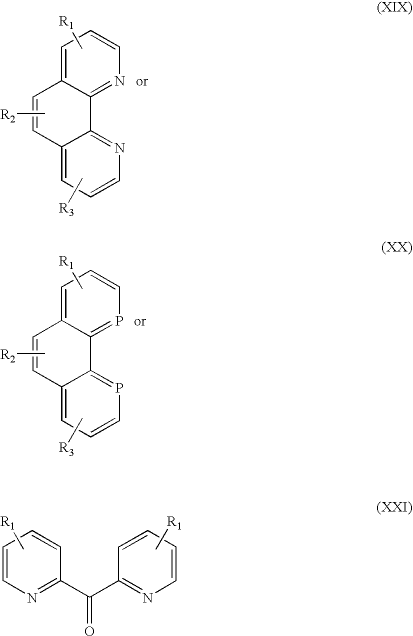

where R₁, R₂ and R₃ are as referred to above, for example bathophen shown in FIG. 3 of the drawings in which R is as above or

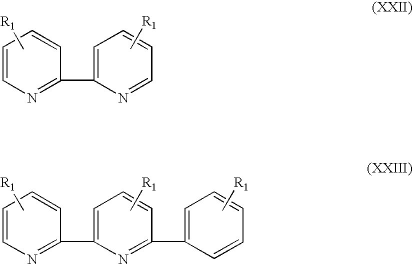

where R₁, R₂ and R₃ are as referred to above.

Lₚ can also be

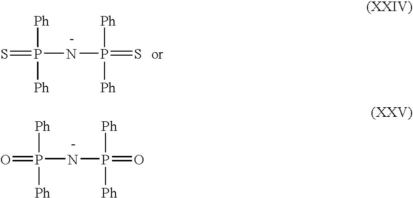

where Ph is as above.

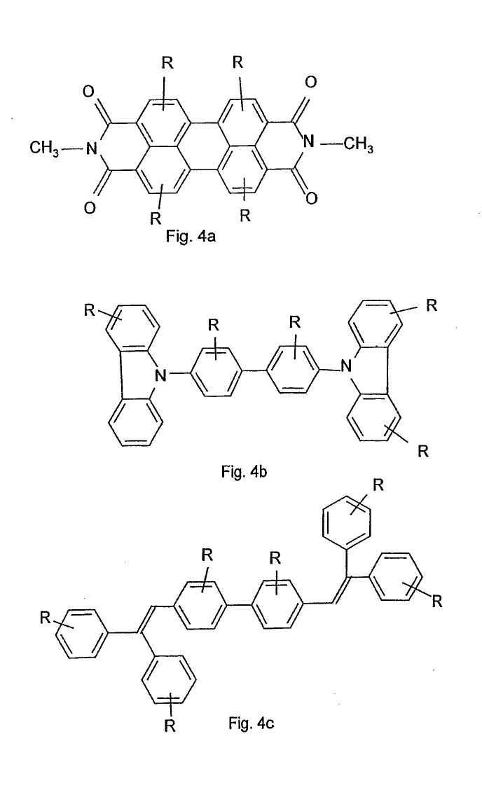

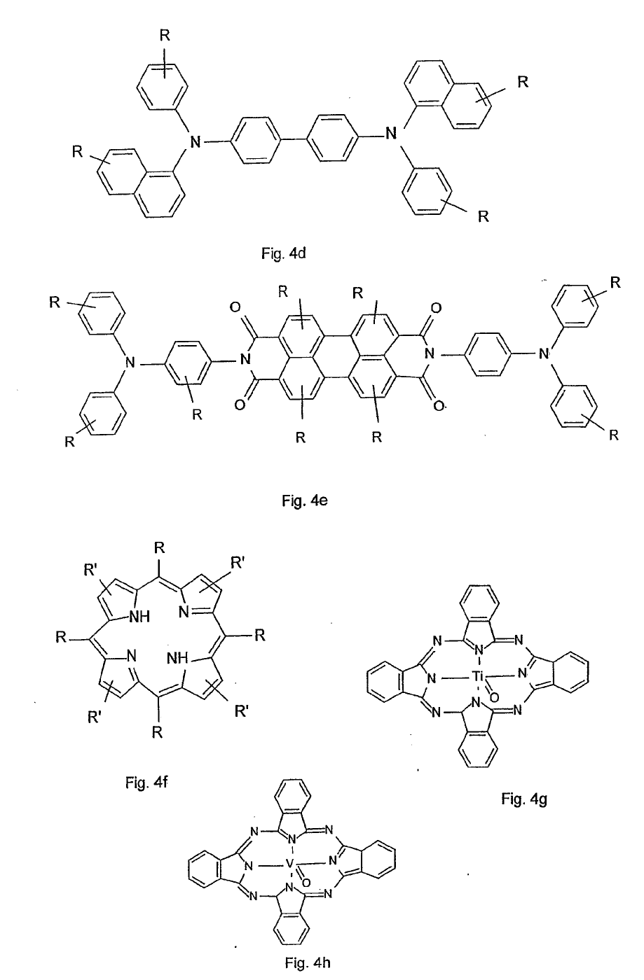

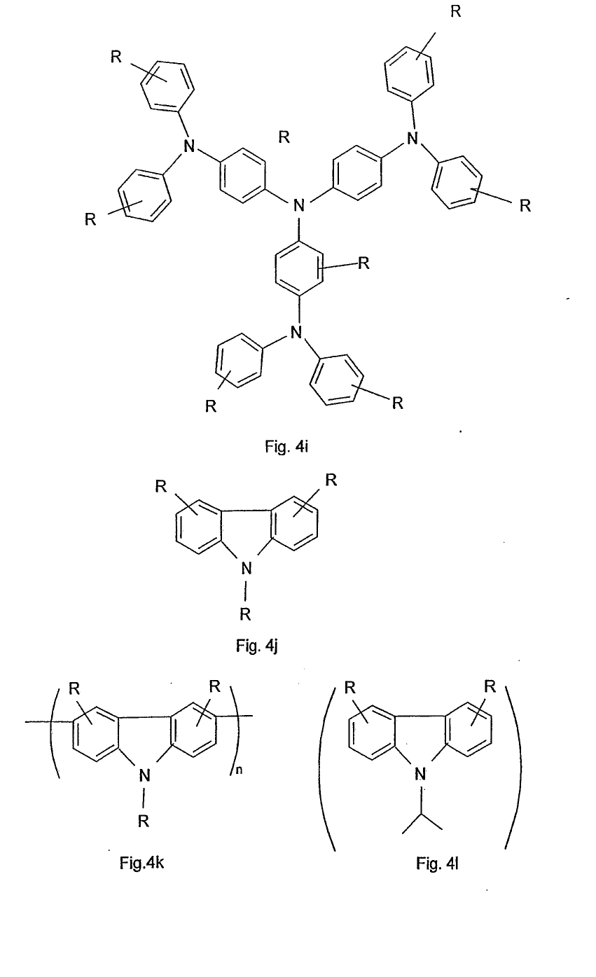

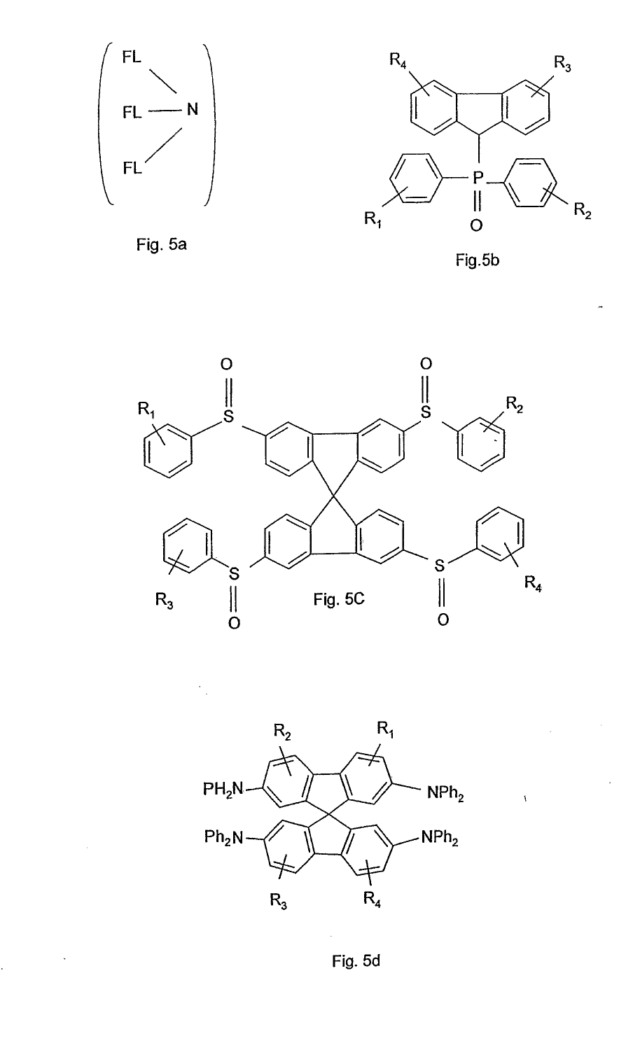

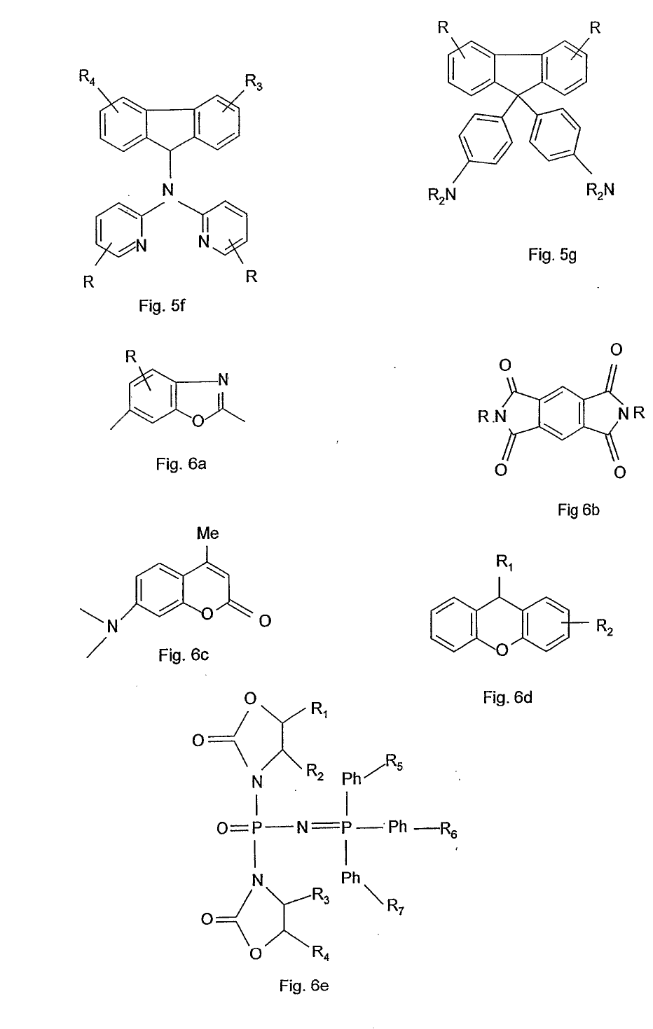

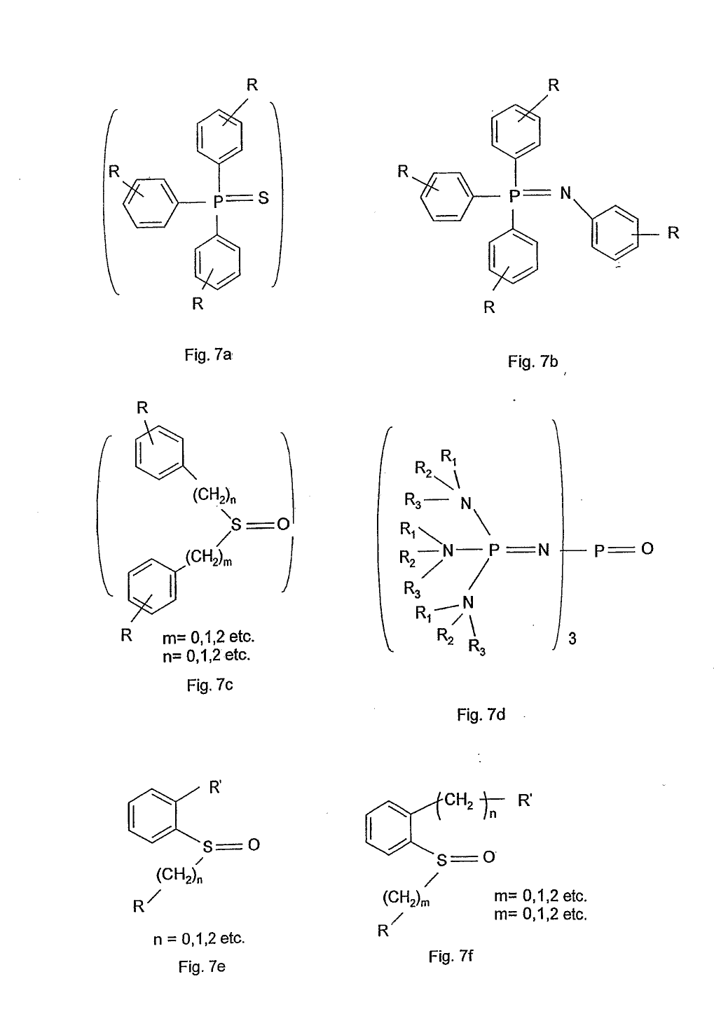

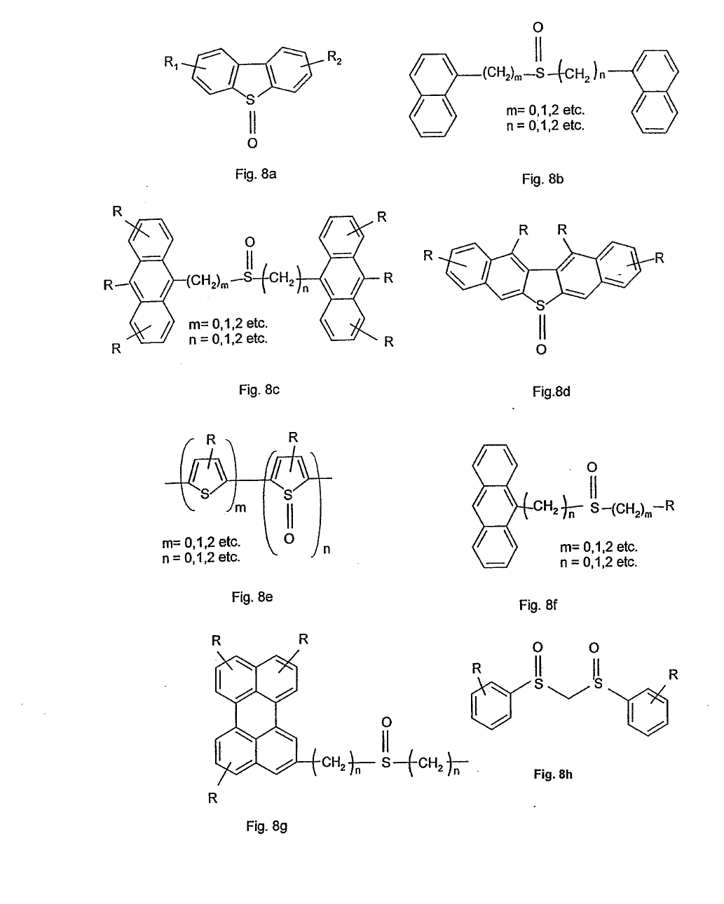

Other examples of Lₚ chelates are as shown in FIG. 4 and fluorene and fluorene derivatives e.g. as shown in FIG. 5 and compounds of formulae as shown in FIGS. 6 to 8.

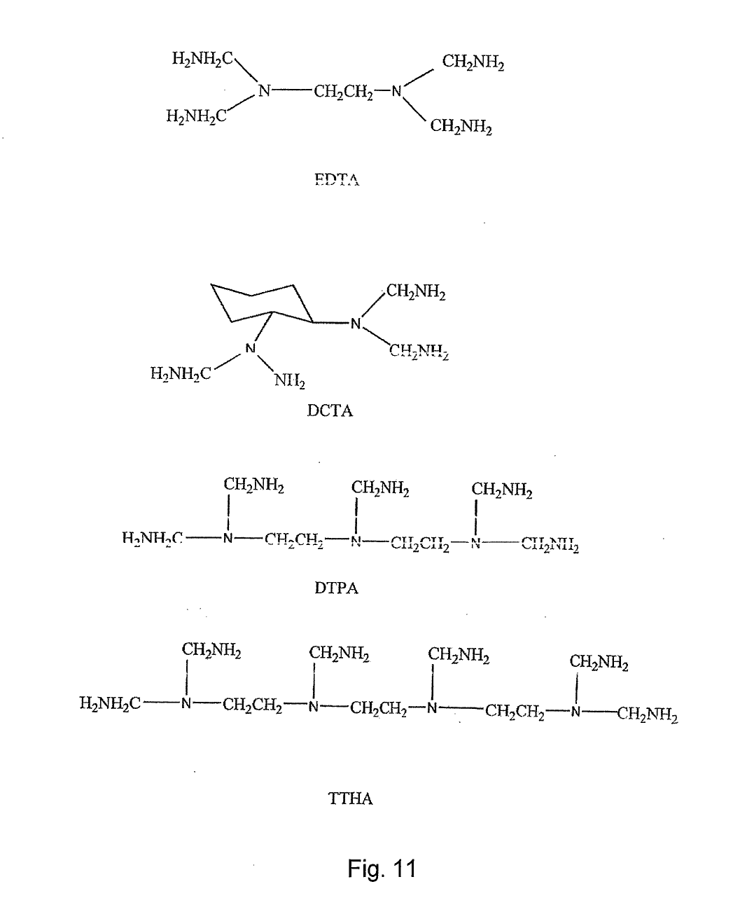

Specific examples of Lα and Lp are tripyridyl and TMHD, and TMHD complexes, α, α′, α″ tripyridyl, crown ethers, cyclans, cryptans phthalocyanans, porphoryins ethylene diamine tetramine (EDTA), DCTA, DTPA and TTHA, where TMHD is 2,2,6,6-tetramethyl-3,5-heptanedionato and OPNP is diphenylphosphonimide triphenyl phosphorane. The formulae of the polyamines are shown in FIG. 11.

Other organic electroluminescent materials which can be used include:—

(1) metal quinolates such as lithium quinolate, and non rare earth metal complexes such as aluminium, magnesium, zinc, zirconium and scandium complexes such as complexes of β-diketones e.g. Tris-(1,3-diphenyl-1-3-propanedione) (DBM) and suitable metal complexes are Al(DBM)₃, Zn(DBM)₂ and Mg(DBM)₂, Sc(DBM)₃ etc.
(2) the metal complexes of formula

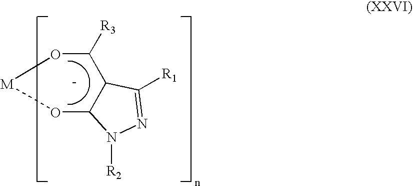

where M is a metal other than a rare earth, a transition metal, a lanthanide or an actinide; n is the valency of M; R₁, R₂ and R₃ which may be the same or different are selected from hydrogen, hydrocarbyl groups, substituted and unsubstituted aliphatic groups substituted and unsubstituted aromatic, heterocyclic and polycyclic ring structures, fluorocarbons such as trifluoryl methyl groups, halogens such as fluorine or thiophenyl groups or nitrile; R₁, and R₃ can also be form ring structures and R₁, R₂ and R₃ can be copolymerisable with a monomer e.g. styrene. Preferably M is aluminium and R₃ is a phenyl or substituted phenyl group.
(3) diiridium complexes of formula

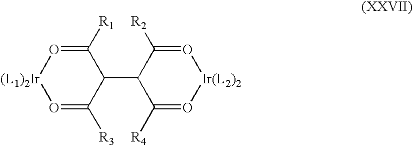

where R₁, R₂, R₃ and R₄ can be the same or different and are selected from hydrogen, and substituted and unsubstituted hydrocarbyl groups.
(4) boron complexes of formula

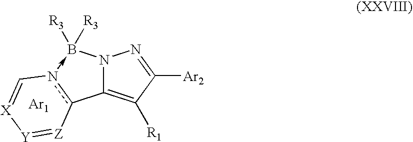

wherein Ar₁ represents a group selected from unsubstituted and substituted monocyclic or polycyclic heteroaryls having a ring nitrogen atom for forming a coordination bond to boron as indicated and optionally one or more additional ring nitrogen atoms subject to the proviso that nitrogen atoms do not occur in adjacent positions, X and Z being selected from carbon and nitrogen and Y being carbon or optionally nitrogen if neither of X and Z is nitrogen, said substituents if present being selected from substituted and unsubstituted hydrocarbyl, substituted and unsubstituted hydrocarbyloxy, fluorocarbon, halo, nitrile, amino alkylamino, dialkylamino or thiophenyl;
Ar₂ represents a group selected from monocyclic and polycyclic aryl and heteroaryl optionally substituted with one or more substituents selected from substituted and unsubstituted hydrocarbyl, substituted and unsubstituted hydrocarbyloxy, fluorocarbon, halo, nitrile, amino, alkylamino, dialkylamino and thiophenyl;
R₁ represents hydrogen or a group selected from substituted and unsubstituted hydrocarbyl, halohydrocarhyl and halo; and
R₂ and R₃ each independently represent a moiety selected from alkyl, cycloalkyl, cycloalkylalkyl, haloalkyl, halo and monocyclic, polycyclic, aryl, heteroaryl, aralkyl and heteroaralkyl optionally substituted with one or more of a moiety selected from alkyl, cycloalkyl, cycloalkylalkyl, haloalkyl, aryl, aralkyl, alkoxy, aryloxy, halo, nitric, amino, alkylamino and dialkylamino.
(5) complexes of formula

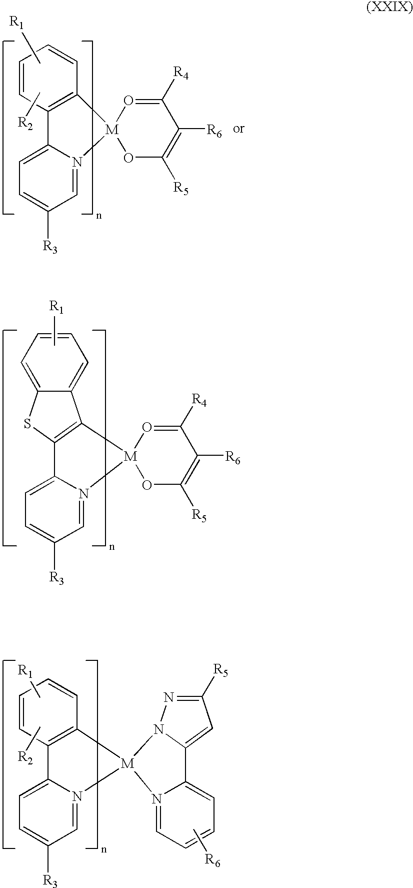

where R₁, R₂, R₃, R₄, R₅ and R₆ can be the same or different and are selected from hydrogen, and substituted and unsubstituted hydrocarbyl groups such as substituted and unsubstituted aliphatic groups, substituted and unsubstituted aromatic, heterocyclic and polycyclic ring structures, fluorocarbons such as trifluoryl methyl groups, halogens such as fluorine or thiophenyl groups; R₁, R₂ and R₃ can also form substituted and unsubstituted fused aromatic, heterocyclic and polycyclic ring structures and can be copolymerisable with a monomer, e.g. styrene, and where R₄, and R₅ can be the same or different and are selected from hydrogen, and substituted and unsubstituted hydrocarbyl groups such as substituted and unsubstituted aliphatic groups, substituted and unsubstituted aromatic, heterocyclic and polycyclic ring structures, fluorocarbons such as trifluoryl methyl groups, halogens such as fluorine or thiophenyl groups; R₁, R₂ and R₃ can also form substituted and unsubstituted fused aromatic, heterocyclic and polycyclic ring structures and can be copolymerisable with a monomer, M is ruthenium, rhodium, palladium, osmium, iridium or platinum and when the valency of M is 2, n is 1, when the valency of M is 3 n is 2 and when the valency of M is 4 n is 3.
(6) complexes of formula

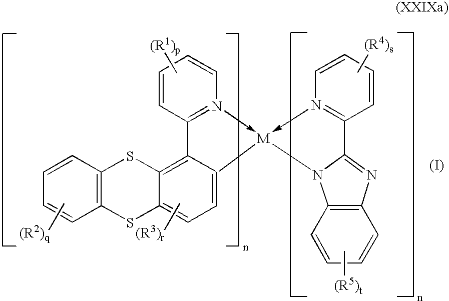

wherein M is ruthenium, rhodium, palladium, osmium, iridium or platinum; n is 1 or 2; R¹, R⁴ and R⁵ can be the same or different and are selected from substituted and unsubstituted hydrocarbyl groups; substituted and unsubstituted monocyclic and polycyclic heterocyclic groups; substituted and unsubstituted hydrocarbyloxy or carboxy groups; fluorocarbyl groups; halogen; nitrile; amino; alkylamino; dialkylamino; arylamino; diarylamino; and thiophenyl; p, s and t independently are 0, 1, 2 or 3; subject to the proviso that where any of p, s and t is 2 or 3 only one of them can be other than saturated hydrocarbyl or halogen; R² and R³ can be the same or different and are selected from; substituted and unsubstituted hydrocarbyl groups; halogen; q and r independently are 0, 1 or 2 and
(7) complexes of formula

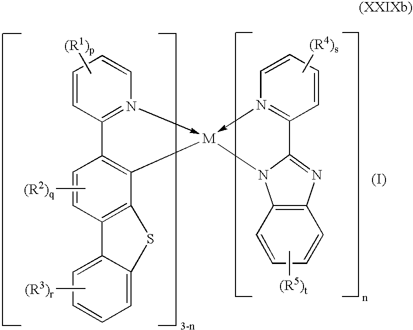

wherein M is ruthenium, rhodium, palladium, osmium, iridium or platinum; n is 1 or 2; R¹-R⁵ which may be the same or different are selected from substituted and unsubstituted hydrocarbyl groups; substituted and unsubstituted monocyclic and polycyclic heterocyclic groups; substituted and unsubstituted hydrocarbyloxy or carboxy groups; fluorocarbyl groups; halogen; nitrile; nitro; amino; alkylamino; dialkylamino; arylamino; diarylamino; N-alkylamido, N-arylamido, sulfonyl and thiophenyl; and R² and R³ can additionally be alkylsilyl or arylsilyl; p, s and t independently are 0, 1, 2 or 3; subject to the proviso that where any of p, s and t is 2 or 3 only one of them can be other than saturated hydrocarbyl or halogen; q and r independently are 0, 1 or 2, subject to the proviso that when q or r is 2, only one of them can be other than saturated hydrocarbyl or halogen,
(8) complexes of formula

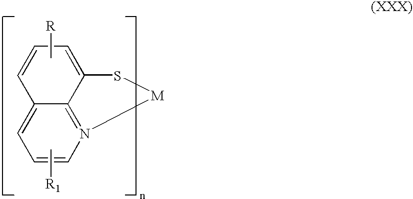

where M is a metal; n is the valency of M; R and R₁ which can be the same or different are selected from hydrogen, and substituted and unsubstituted hydrocarbyl groups such as substituted and unsubstituted aliphatic groups, substituted and unsubstituted aromatic, heterocyclic and polycyclic ring structures, fluorocarbons such as trifluoryl methyl groups, halogens such as fluorine; thiophenyl groups; cyano group; substituted and unsubstituted hydrocarbyl groups such as substituted and unsubstituted aliphatic groups, substituted and unsubstituted aliphatic groups as described in patent application PCT/GB2005/002579.

In another electroluminescent structure the electroluminescent layer is formed of layers of two electroluminescent organic complexes in which the band gap of the second electroluminescent metal complex or organo metallic complex such as a gadolinium or cerium complex is larger than the band gap of the first electroluminescent metal complex or organo metallic complex such as a europium or terbium complex.

Electroluminescent materials and devices are described in patent applications PCT/GB98/01773, PCT/GB99/03619, PCT/GB99/04030, PCT/GB99/04024, PCT/GB99/04028, PCT/GB00/00268, PCT/GB01/05113, PCT/GB01/05111, PCT/GB01/05135, PCT/GB021264, PCT/GB02/01837, PCT/GB02/018884, PCT/GB02/01839, PCT/GB02/01844, PCT/GB02/02094 PCT/GB02/02092 and PCT/GB02/02093 the contents of which are incorporated by reference.

Polymer electroluminescent materials which can be used are semiconductive and/or conjugated polymer materials. Alternatively the light-emissive material could be of other types, for example sublimed small molecule films or inorganic light-emissive material. The organic, or each organic light-emissive material may comprise one or more individual organic materials, suitably polymers, preferably fully or partially conjugated polymers. Example materials include one or more of the following in any combination: poly(p-phenylenevinylene) (“PPV”), poly(2-methoxy-5(2′-ethyl)hexyloxyphenylene-vinylene) (“MEH-PPV”), one or more PPV-derivatives (e.g. di-alkoxy or di-alkylderivatives), polyfluorenes and/or co-polymers incorporating polyfluorenesegments, PPVs and related co-polymerst poly(217-(9,9-di-n-octylfluorene)-(1,4-phenylene-((4-secbutylphenyl)imino)-1,4-phenylene)) (“TFB”), poly(2,7-(9,9-di-n-octylfluorene)-(1 4-phenylene-((4-methylphenyl)imino)-1 4-phenylene-((4-methylphenyl)imino)-1,4-phenylene)) (“PFM”), poly(2,7-(919-di-n-octylfluorene)(1 4-phenylene-((4-methoxyphenyl)imino)-1,4-phenylene-((4-methoxyphenyl)imino)-1,4-phenylene)) (“PFIVIO”), poly(2,7-(9,9-di-n-octylfluorene) (“F8”) or (2,7-(9,9-di-n-octylfluorene)-3,6-Benzothiadiazole) (“RBT”). Alternative materials include small molecule materials such as aluminium quinolate (Alq3).

Optionally the hole transporting material can be mixed with the electroluminescent material and co-deposited with it.

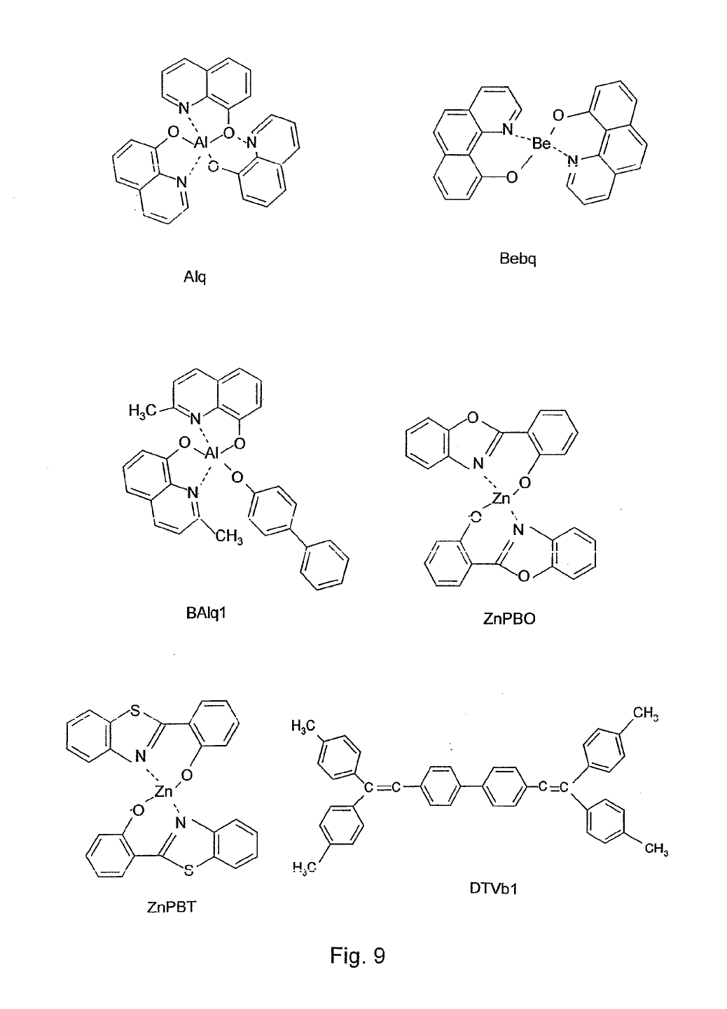

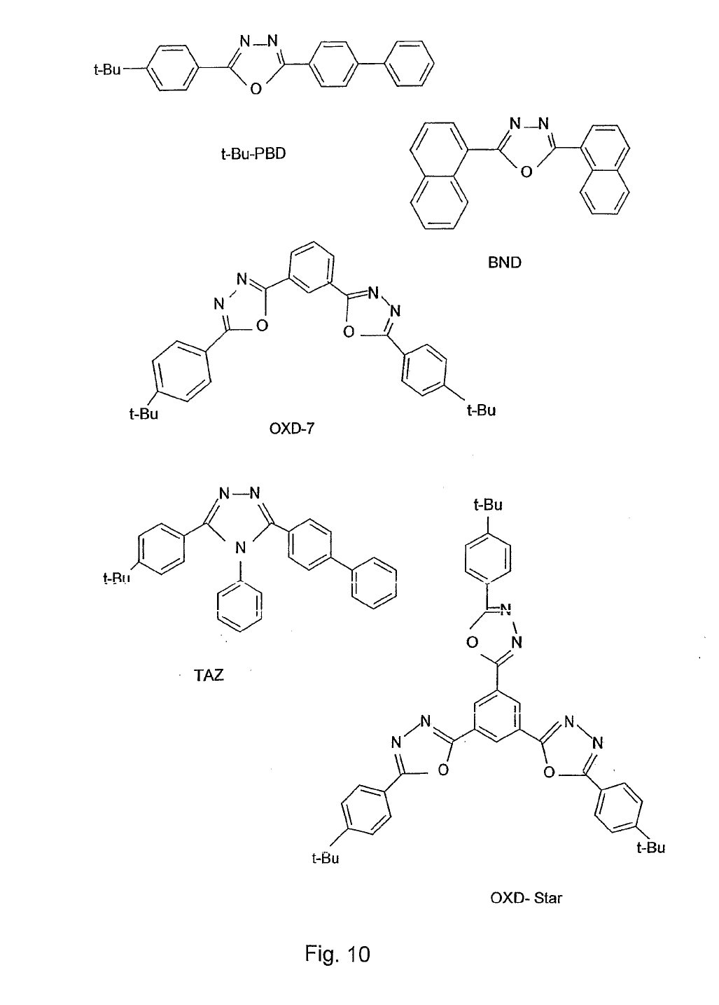

Optionally there is a layer of an electron injecting material between the cathode and the electroluminescent composition layer; the electron injecting material is a material which will transport electrons when an electric current is passed through electron injecting materials and include a metal complex such as a metal quinolate or thioquinolate e.g. an aluminium quinolate, lithium quinolate, zirconium quinolate, indium thioquinolate, gallium thioquinolate; a compound of formula Mx(DBM)ₙ where Mx is a metal and DBM is dibenzoyl methane and n is the valency of Mx, e.g. Mx is chromium. The electron injecting material can also be a cyano anthracene such as 9,10 dicyano anthracene, cyano substituted aromatic compounds, tetracyanoquinidodimethane, a polystyrene sulphonate or a compound with the structural formulae shown in FIG. 9 or 10 of the drawings in which the phenyl rings can be substituted with substituents R as defined above; or a metal thioxinate of formula (XXX).

Instead of being a separate layer the electron injecting material can be mixed with the electroluminescent material and co-deposited with it.

The hole transporting materials, the electroluminescent material and the electron injecting materials can be mixed together to form one layer, which simplifies the construction.

The first electrode is preferably a transparent substrate such as a conductive glass or plastic material which acts as the anode. Preferred substrates are conductive glasses such as indium tin oxide coated glass, but any glass which is conductive or has a conductive layer such as a metal or conductive polymer can be used. Conductive polymers and conductive polymer coated glass or plastics materials can also be used as the substrate.

The cathode is preferably a low work function metal, e.g. aluminium, calcium, lithium, magnesium and alloys thereof such as silver/magnesium alloys, rare earth metal alloys etc; aluminium is a preferred metal. A metal fluoride such as an alkali metal, rare earth metal or their alloys can be used as the second electrode, for example by having a metal fluoride layer formed on a metal.

The devices of the present invention can be used as displays in video displays, mobile telephones, portable computers and any other application where an electronically controlled visual image is used. The devices of the present invention can be used in both active and passive applications of such as displays.

In known electroluminescent devices either one or both electrodes can be formed of silicon and the electroluminescent material and intervening layers of hole transporting and electron transporting materials can be formed as pixels on the silicon substrate.

Preferably each pixel comprises at least one layer of an electroluminescent material and a (at least semi-) transparent electrode in contact with the organic layer on a side thereof remote from the substrate.

Preferably, the substrate is of crystalline silicon and the surface of the substrate may be polished or smoothed to produce a flat surface prior to the deposition of electrode, or electroluminescent compound. Alternatively a non-planarised silicon substrate can be coated with a layer of conducting polymer to provide a smooth, flat surface prior to deposition of further materials.

In one embodiment, each pixel comprises a metal electrode in contact with the substrate. Depending on the relative work functions of the metal and transparent electrodes, either may serve as the anode with the other constituting the cathode.

When the silicon substrate is the cathode an indium tin oxide coated glass can act as the anode and light is emitted through the anode. When the silicon substrate acts as the anode, the cathode can be formed of a transparent electrode which has a suitable work function; for example by an indium zinc oxide coated glass in which the indium zinc oxide has a low work function. The anode can have a transparent coating of a metal formed on it to give a suitable work function. These devices are sometimes referred to as top emitting devices or back emitting devices.

The metal electrode may consist of a plurality of metal layers; for example a higher work function metal such as aluminium deposited on the substrate and a lower work function metal such as calcium deposited on the higher work function metal. In another example, a further layer of conducting polymer lies on top of a stable metal such as aluminium.

Preferably, the electrode also acts as a mirror behind each pixel and is either deposited on, or sunk into, the planarised surface of the substrate. However, there may alternatively be a light absorbing black layer adjacent to the substrate.

In still another embodiment, selective regions of a bottom conducting polymer layer are made non-conducting by exposure to a suitable aqueous solution allowing formation of arrays of conducting pixel pads which serve as the bottom contacts of the pixel electrodes.

### EXAMPLES

### Example 1

### Preparation of 1:1 [9,9′]Bianthracenyl/Toluene adduct

Anthrone (40.00 g, 206 mmol) was refluxed in a mixture of glacial acetic acid (200 ml) and concentrated hydrochloric acid (80 ml). To this refluxing solution granulated tin (80 g, 674 mmol) was cautiously added. The reaction was refluxed for 15 h during which time a white precipitate formed. The mixture was cooled to room temperature and the solution was carefully filtered under vacuum to isolate the precipitate but left unreacted in the reaction vessel. The precipitate was washed with water (100 ml) and dried in a vacuum oven. This solid was then recrystallised from the minimum amount of hot toluene (approximately 500 ml) to yield light yellow crystals of the 1:1 [9,9′]Bianthracenyl/Toluene adduct (37 g, 81% yield).

### Example 2

### Preparation of 10:10′dibromo-[9,9′]bianthracenyl

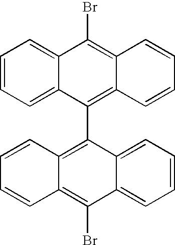

### 10:10′dibromo-[9,9′]bianthracenyl

to a solution of 1:1 [9,9′]Bianthracenyl/Toluene adduct in carbon disulfide (100 ml) at room temperature bromine (6.9 ml, 2 34.7 mmol) was added drop wise. Hydrogen bromide fumes were evolved and the mixture was stirred for a further 2 h. After this period n-(Hexane (150 ml) was added and a large amount of yellow solid precipitated. This solid was filtered under vacuum, washed with n-Hexane and dried. This solid was 10:10′dibromo-[9,9′]bianthracenyl (27 g, 78%); m.p. 357-359° C.

### Example 3

### Preparation of Biphenyl-4-yl-m-tolyl-amine

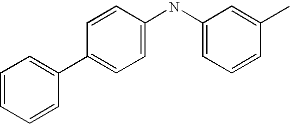

### Biphenyl-4-yl-m-tolyl-amine

4-Bromobiphenyl (15.0 g, 64.4 mmol), Sodium tert-Butoxide (6.9 g, 71.8 mmol), Dichloro[1,1′-bis(diphenylphosohino)ferrocene]palladium (II) dichloromethane adduct (0.47 g, 0.64 mmol) and 1,1′-Di(Diphenylphosphano)ferrocene (1.07 g. 1.93 mmol) were dissolved in dry o-Xylene (100 ml) and stirred under an atmosphere of dry Argon gas. To this solution was added m-Toluidine (7 ml, 65.3 mmol) via a syringe/septum. The solution became very dark. The mixture was heated at 120° C. for 3 h over which time the mixture became a light cloudy orange. The mixture poured in to a conical flask, heated with 100 ml of toluene and filtered whilst hot to remove the white inorganic residues. The solvent was removed under vacuum and ethanol was added to the remaining liquid. This mixture was cooled in a fridge overnight and a crystalline solid formed. This solid was filtered and washed with a small amount of cold ethanol. The solid was Biphenyl-4-yl-m-tolyl-amine and was pure enough for use in synthesis. (12 g, 72%); m.p. 95° C.

### Example 4

### General synthesis for 10,10′diamino-[9,9′]dianthracenyl materials. (A)

This is a general synthesis for these materials; in each separate case a different diarylamine is utilised and a different workup procedure utilized

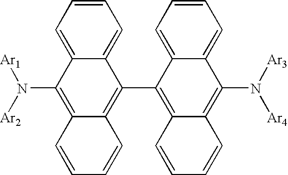

### 10,10′diamino-[9,9′]dianthracenyl materials

10,10′-Dibromo-[9,9′]bianthracenyl (2.0 g, 3.9 mmol), Diarylamine (7.8 mmol), Sodium tert-butoxide (0.83 g, 8.64 mmol), Palladium(II)acetate (0.09 g, 04 mmol) and tri-tert-butyl-phosphane 10% wt in hexane (5.4 ml, 16 mmol) were stirred in dry o-Xylene (20 ml) under an atmosphere of dry Argon gas. This mixture was heated to 120° C. for 2 h. The initial dark solution became lighter with a precipitate over this period. The reaction mixture was cooled to room temperature. The particular workup is noted with each different compound.

### Example 5

### Preparation of N*10*,N*10′*Bis-biphenyl-4-yl-N*10*,N*10′*-di-m-toly-[9,9′]bianthracenyl-10,10′-diamine. (B)

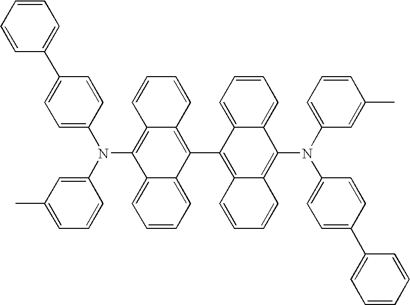

### N*10*,N*10′*Bis-biphenyl-4-yl-N*10*,N*10′*-di-m-toly-[9,9′]bianthracenyl-10,10′-diamine

Biphenyl-4-yl-m-tolyl-amine was used as the starting diarylamine.

Workup: The reaction solution was heated with toluene (50 ml) and filtered. The solution was evaporated to dryness and the residue recrystallised thrice from THF/Methanol and dried in a vacuum oven.

### Example 6

### Preparation of N*10*,N*10′*-Di-naphthen-yl-N*10*,N*10*-di-m-toly-[9,9′]bianthracenyl-10,10′-diamine. (C)

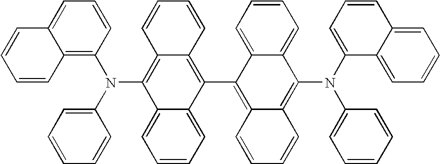

### N*10*,N*10′*-Di-naphthen-yl-N*10*,N*10*-di-m-toly-[9,9′]bianthracenyl-10,10′-diamine

N-Phenyl-1-naphthylamine was used as the starting diarylamine.

Workup: The reaction solution was heated with toluene (50 ml) and filtered. The solution was evaporated to dryness and the residue recrystallised thrice from THF/Methanol and dried in a vacuum oven.

### Example 7

### Preparation of N*10*,N*10′*-Di-phenyl-N*10*,N*10′*-di-m-toly-[9,9′]bianthracenyl-10,10′-diamine. (D)

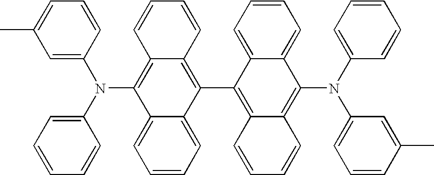

### N*10*,N*10′*Di-phenyl-N*10*,N*10′*-di-m-toly-[9,9′]bianthracenyl-10,10′-diamine

3-Methyldiphenylamine was used as the starting diarylamine.

Workup: The reaction mixture was cooled to room temperature and evaporated to dryness. The residue was, dissolved in hot THF (100 ml) and filtered. To the cooled THF solution was added Methanol (200 ml), which caused a green/yellow precipitate to form. This precipitate was filtered and dried. The solid was dissolved in THF and precipitated with Methanol. The precipitate was filtered and dried in a vacuum oven.

### Example 8

### Preparation of N*10*,N*10′*-Bis-(3-methoxy-phenyl)-N*10*,N*10′*-diphenyl-[9,9′]bianthracenyl-10,10′-diamine. (E)

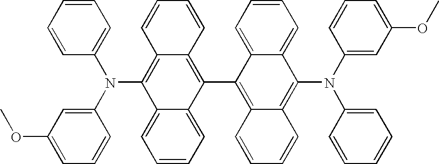

### N*10*,N*10′*-Bis-(3-methoxy-phenyl)-N*10*,N*10′*-diphenyl-[9,9′]bianthracenyl-10,10′-diamine

3-Methoxydiphenylamine was used as the starting diarylamine.

Workup: The reaction mixture was filtered and washed with hot THF. The solid was dried and vacuum sublimed to give a yellow solid.

### Example 9

### Preparation of N*10*,N*10′*-Di-naphthen-2-yl-N*10*,N*10′*-di-phenyl-[9,9′]bianthracenyl-10,10′-diamine. (F)

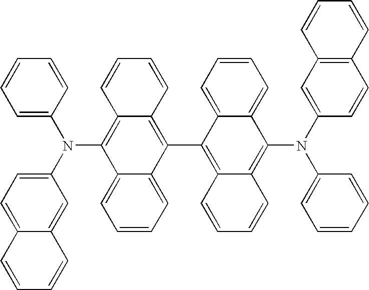

### N*10*,N*10′*-Di-naphthen-2-yl-N*10*,N*10′*-di-phenyl-[9,9′]bianthracenyl-10,10′-diamine

N-Phenyl-2-Naphthylamine was used as the starting diarylamine.

Workup: The reaction mixture was filtered and washed with hot THF. The solid was dried and vacuum sublimed to give an orange solid.

### Example 10

### Preparation of

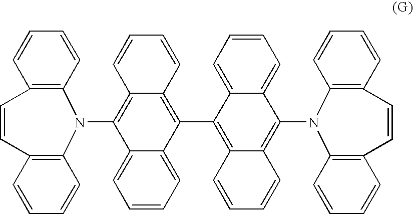

Iminostilbene was used as the starting diarylamine.

Workup: The reaction mixture was filtered and washed with toluene (100 ml) then ethanol (100 ml) then water (100 ml) then ethanol (100 ml) then dried. The solid was then recrystallised from DCM/Hexane to give light orange micro crystals.

It is a feature of the hole transporting complexes of the present invention that they have improved thermal stability compared with other hole transporting compounds which makes them more useful in electroluminescent devices.

The properties of the compounds were measured and results shown in the Table.

TABLE

| | | | Optical |
|---|---|---|---|
| Compound | Thermal Data | Elemental | Band Gap |
| 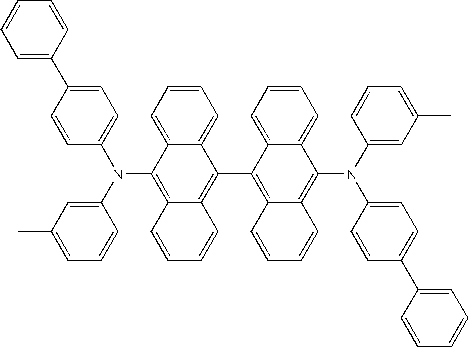 | Tm: >400° C.Tg: 192° C. | % Theory C: 91.21 H: 5.57 N: 3.22% Found C: 90.28 H: 5.34 N: 2.64 | |
| 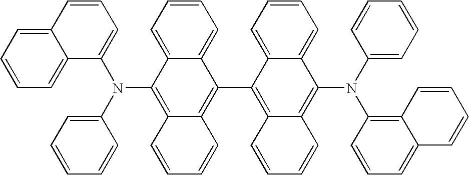 | Tm: >400° C.Tg: 220° C. | % Theory C: 91.34 H: 5.11 N: 3.55% Found C: 90.78 H: 4.99 N: 3.29 | 2.56eV |
| 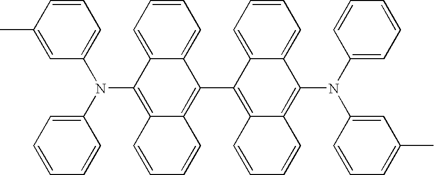 | Tm: 392-396° C. | % Theory C: 90.47 H: 5.62 N: 3.91% Found C: 90.19 H: 5.53 N: 3.75 | 2.53eV |
| 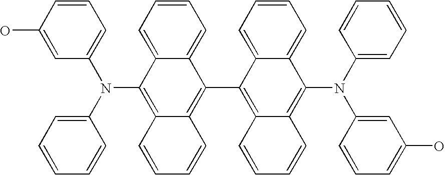 | Tm: 395-401° C. | % Theory C: 86.60 H: 5.68 N: 3.74% Found C: 86.68 H: 5.30 N: 3.75 | 2.56eV |
| 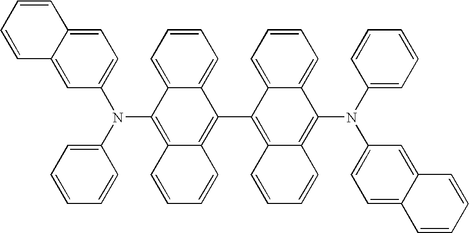 | Tm: ~420° C.Tg: 196° C. | % Theory C: 91.34 H: 5.11 N: 3.55% Found C: 91.13 H: 5.02 N: 3.55 | |
| 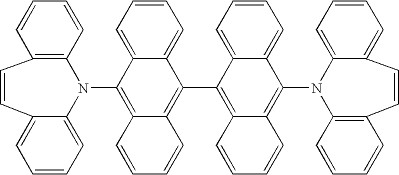 | Tm: >420° C. | % Theory C: 91.27 H: 4.92 N: 3.80% Found C: 90.44 H: 4.90 N: 3.74 | 2.88eV |

### Electroluminescent Devices

### Example 11

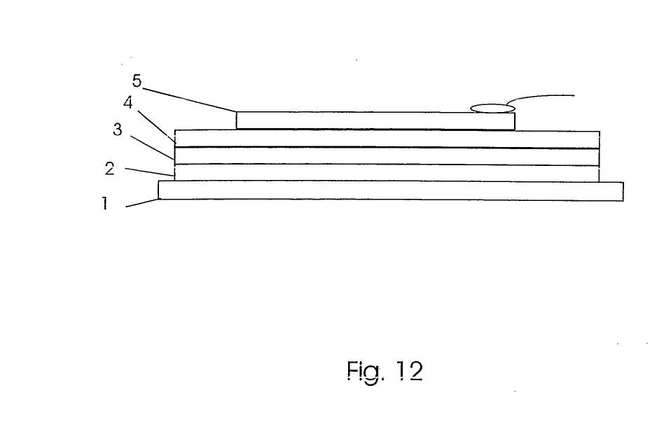

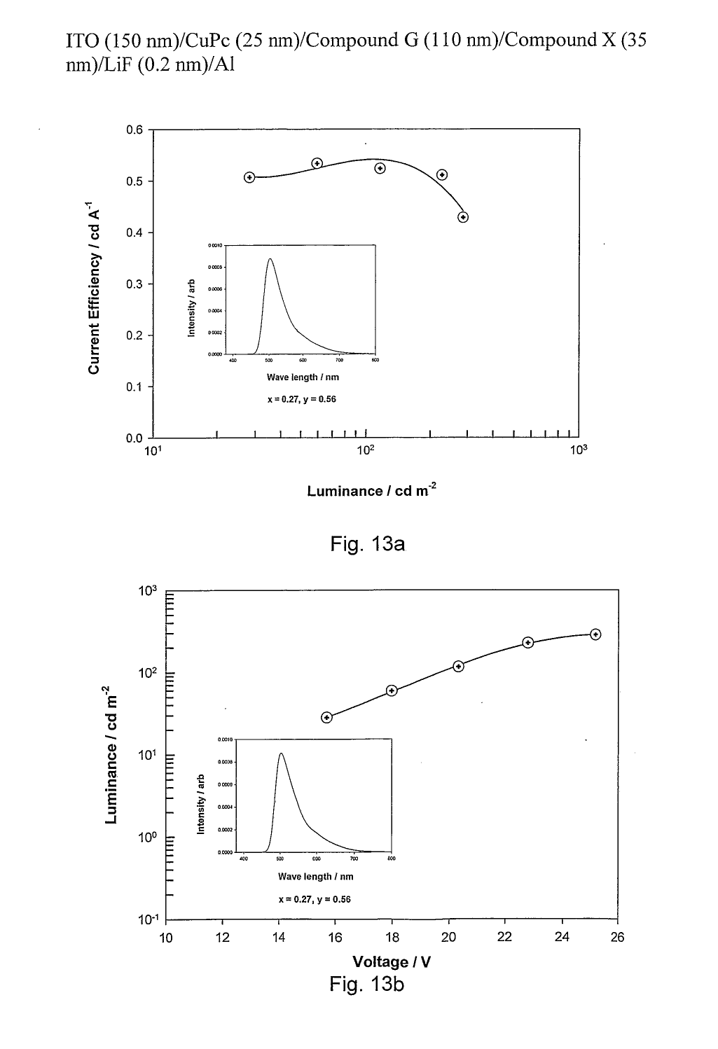

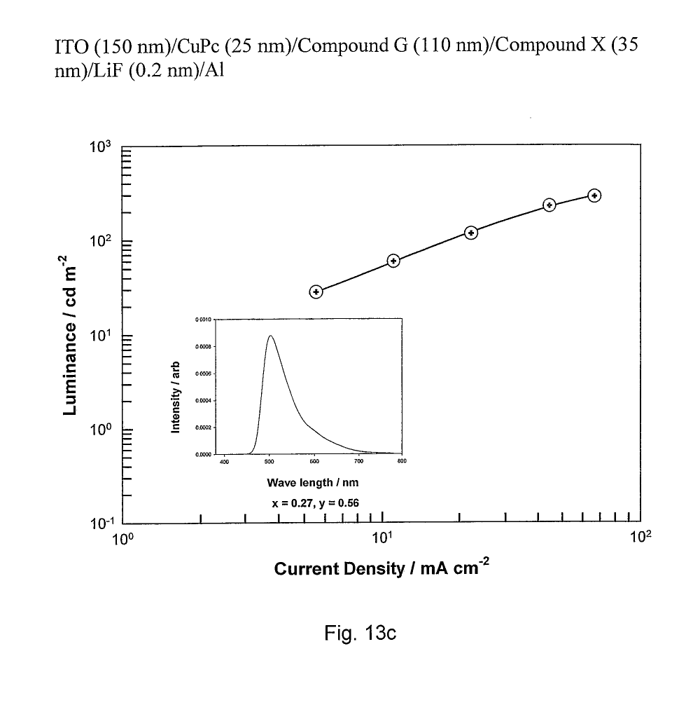

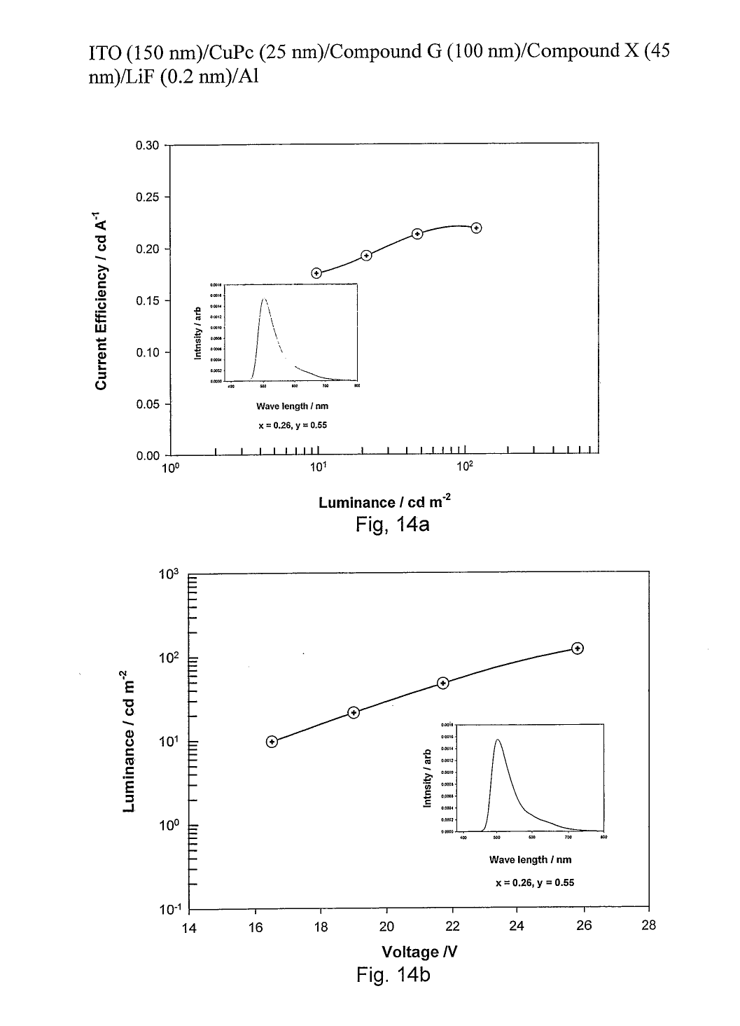

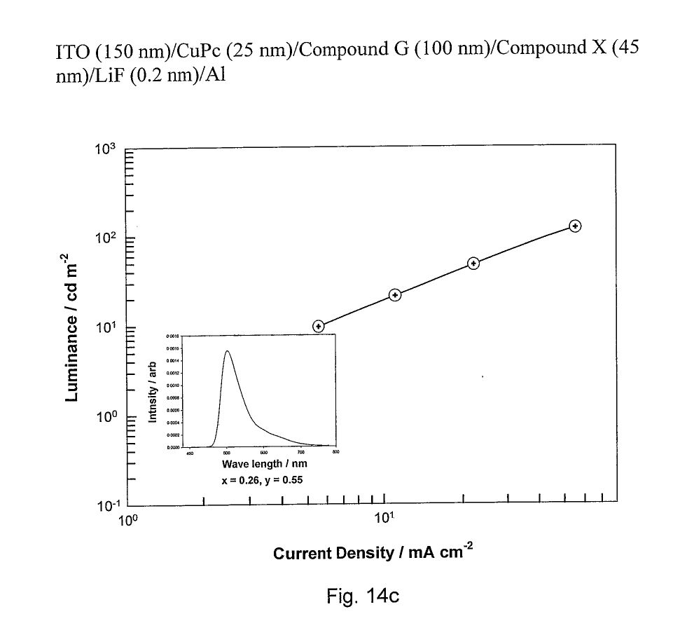

A pre-etched ITO coated glass piece (10×10 cm²) was used. The device was fabricated by sequentially forming on the ITO, by vacuum evaporation the compositions forming the layers comprising the electroluminescent device. The layers were deposited using a Solciet Machine, ULVAC Ltd. Chigacki, Japan. The active area of each pixel was 3 mm by 3 mm; the device is shown in FIG. 12 and the layers comprised:—

(1)ITO (150 nm)/(2)CuPc (25 nm)/(3)Compound G (110 nm)/(4)Compound X (35 nm)/(5)LiF (0.2 nm)/Al
where ITO is indium tin oxide coated glass, CuPc is copper phthalocyanine and compound G is as in Example 10 and compound X is as shown below.

The coated electrodes were stored in a vacuum desiccator over a molecular sieve and phosphorous pentoxide until they were loaded into a vacuum coater (Edwards, 10⁻⁶ torr) and aluminium top contacts made. The devices were then kept in a vacuum desiccator until the electroluminescence studies were performed.

The ITO electrode was always connected to the positive terminal. The current vs. voltage studies were carried out on a computer controlled Keithly 2400 source meter.

A voltage was applied across the device and the properties measured and the results are shown in FIGS. 9a, 9b and 9c.

### Example 12

A device was formed as in Example 10 with the structure:—

ITO (150 nm)/CuPc (25 nm)/Compound G (100 nm)/Compound X (45 nm)/LiF (0.2 nm)/Al
where Compounds G and X are as in Example 11.

A voltage was applied across the device and the properties measured and the results are shown in FIGS. 10a, 10b and 10c.

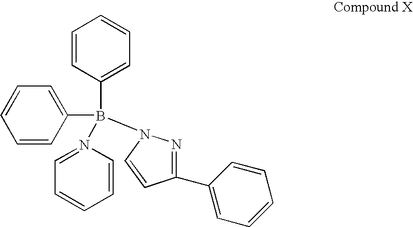

1-28. (canceled)

29. A hole transport compound which is a diamino dianthracene of formula 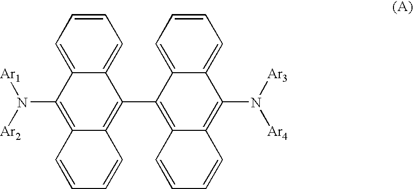
where Ar₁, Ar₂, Ar₃ and Ar₄ are the same or different substituted or unsubstituted aromatic groups or together form a heterocyclic ring with the nitrogen atom.

30. The compound of claim 29 in which:
the substituted or unsubstituted monocyclic or polycyclic aromatic groups are selected from bisphenyl, naphthyl, anthracenyl, and
substituents can be selected from hydrogen, and alkyl, alkoxy, aryl, aryloxy, heterocyclic and carboxy groups and trifluoromethyl.

31. The compound of claim 29, wherein the groups Ar₁, Ar₂, Ar₃ and Ar₄ are selected from

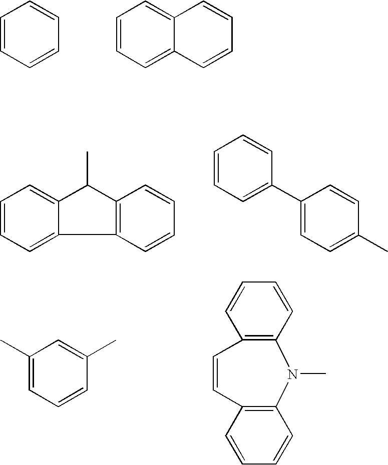

32. Any of the following compounds:
N*10*,N*10′*Bis-biphenyl-4-yl-N*10*,N*10′*-di-m-toly-[9,9′]bianthracenyl-10,10′-diamine;
N*10*,N*10′*-Di-naphthen-yl-N*10*,N*10*-di-m-toly-[9,9′]bianthracenyl-10,10′-diamine;
N*10*,N*10′*-Bis-(3-methoxy-phenyl)-N*10*,N*10′*-diphenyl[9,9′]bianthracenyl-10,10′-diamine;
N*10*,N*10′*-Di-naphthen-2-yl-N*10*,N*10′*-di-phenyl-[9,9′ ]bianthracenyl-10,10′-diamine; and

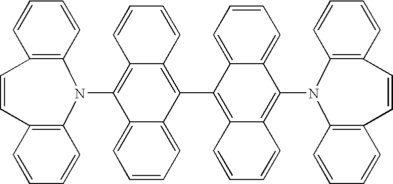

33. A method for making a diamino dianthracene which comprises aminating 10,10′-dibromo-[9,9′]-bianthracene of formula 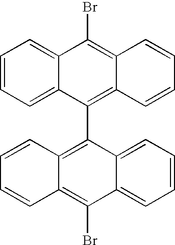
with an amine of formula Ar₁NHAr₂ where Ar₁ and Ar₂ are the same or different substituted or unsubstituted aromatic groups or together with the nitrogen atom to which they are attached form a heterocyclic ring.

34. The method of claim 33, wherein the amination is with an amine of formula Ar₁NHAr₂ where Ar₁ and Ar₂ are different substituted or unsubstituted aromatic groups.

35. The method of claim 33, wherein the amination is with an amine in which the groups Ar₁, Ar₂ are selected from

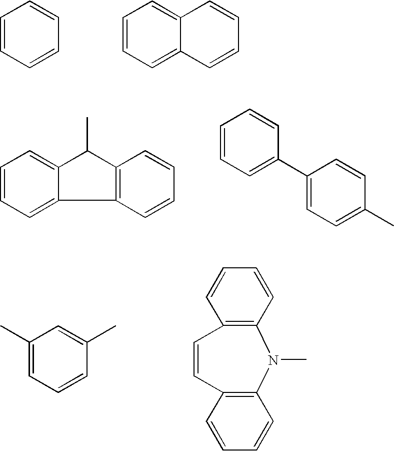

36. The method of any of claim 33 wherein the amination is carried out by heating 10,10′-dibromo-[9,9′]-bianthracene and the amine in the presence of sodium t-butoxide, palladium(II)acetate and tris-t-butyl-phosphane in o-xylene under an atmosphere of dry argon gas.

37. An electroluminescent device which comprises (i) a first electrode, (ii) a layer comprising a hole transport compound as defined in claim 29, (iii) a layer of an electroluminescent material and (iv) a second electrode.

38. The device of claim 37, wherein the electroluminescent material is: a polymer electroluminescent compound.

39. The device of claim 37, wherein the electroluminescent compound is a small molecule organometallic electroluminescent compound.

40. The device of claim 37 wherein the electroluminescent compound is selected from lithium quinolate, aluminium quinolate, zirconium quinolate, hafnium quinolate, gallium thioquinolate and indium thioquinolate.

41. The device claim 37, wherein there is a layer of an electron injecting material between the cathode and the electroluminescent material layer.

42. The device of claim 41, wherein the electron injecting material is selected from metal quinolates, and complexes of formula Mx(DBM)ₙ where Mx is a metal and DBM is dibenzoyl methane and n is the valency of Mx.

43. The device of claim 42 in which the metal quinolate is an aluminium quinolate or lithium quinolate.

44. The device of claim 37, wherein the first electrode is the anode and is indium tin oxide coated glass or another a transparent substrate.

45. The device of claim 44, wherein the cathode is selected from aluminium, calcium, lithium, magnesium and alloys thereof.

46. The device of claim 45, wherein there is a layer of a metal fluoride layer formed on the metal cathode.
